(19) United States
(12) Patent Application Publication
(10) Pub. No.: US 2013/0006152 A1
(43) Pub. Date: Jan. 3, 2013

(54) ELECTRONIC DEVICE AND METHOD FOR ASSISTING CORRECTION OF WALKING STYLES

(75) Inventors: Hou-Hsien LEE, Tu-Cheng (TW); Chang-Jung LEE, Tu-Cheng (TW); Chih-Ping LO, Tu-Cheng (TW)

(73) Assignee: HON HAI PRECISION INDUSTRY CO., LTD., Tu-Cheng (TW)

(21) Appl. No.: 13/472,413

(22) Filed: May 15, 2012

(30) Foreign Application Priority Data
Jun. 29, 2011 (TW) 100122808

(51) Int. Cl. A61B 5/11

(52) U.S. Cl. 600/595

(57) ABSTRACT

An electronic device receives real-time data in relation to plantar pressures of a user. The real-time data is detected by pressure sensors, which is installed within insoles in a pair of shoes of the user, and sent to the electronic device via a wireless communication module installed in the insoles or the shoes. The electronic device then generates a real-time image in relation to real-time plantar pressure distributions of the user according to the real-time data, compares the real-time image with a pre-stored image stored in a storage device, and determines if a comparison result satisfies a preset requirement. The pre-stored image is in relation to standard plantar pressure distributions produced from a standard walking style. If the comparison result does not satisfy the preset requirement, the electronic device displays alert information via an output device to prompt the user to adjust the user's walking style.

### BACKGROUND

1. Technical Field

The embodiments of the present disclosure relate to correction systems and methods, and particularly to an electronic device and a method for assisting correction walking styles of users.

2. Description of Related Art

Walking is a daily fundamental movement and it is frequently chosen as an exercise and may be part of a program for people to improve or maintain health. However, a prolonged and improper style of walking may have detrimental effects on the feet, which is bad for health.

### BRIEF DESCRIPTION OF THE DRAWINGS

FIG. 1 is one embodiment of function modules of an electronic device comprising a control unit for assisting correction of walking styles.

FIG. 2 is a flowchart of one embodiment of a method for assisting correction of walking styles.

FIG. 3 is a figure of one embodiment illustrating insoles in shoes of a user, and a plantar pressure image generated based on data in relation to plantar pressures that are detected by pressure sensors installed within the insoles.

FIG. 4 is one embodiment illustrating how to enable the function for assisting correction of walking styles in the electronic device of FIG. 1.

FIG. 5 is one embodiment illustrating a process of generating a preset plantar pressure distribution image in relation to a standard walking style.

FIG. 6 is one embodiment illustrating a prompt to a user to adjust his walking style by the electronic device in FIG. 1.

### DETAILED DESCRIPTION

The disclosure is illustrated by way of examples and not by way of limitation in the figures of the accompanying drawings in which like references indicate similar elements.

In general, the word “module”, as used herein, refers to logic embodied in hardware or firmware, or to a collection of software instructions, written in a programming language, such as, Java, C, or assembly. One or more software instructions in the modules may be embedded in firmware, such as in an erasable programmable read only memory (EPROM). The modules described herein may be implemented as either software and/or hardware modules and may be stored in any type of non-transitory computer-readable medium or other storage device. Some non-limiting examples of non-transitory computer-readable media include CDs, DVDs, BLU-RAY, flash memory, and hard disk drives.

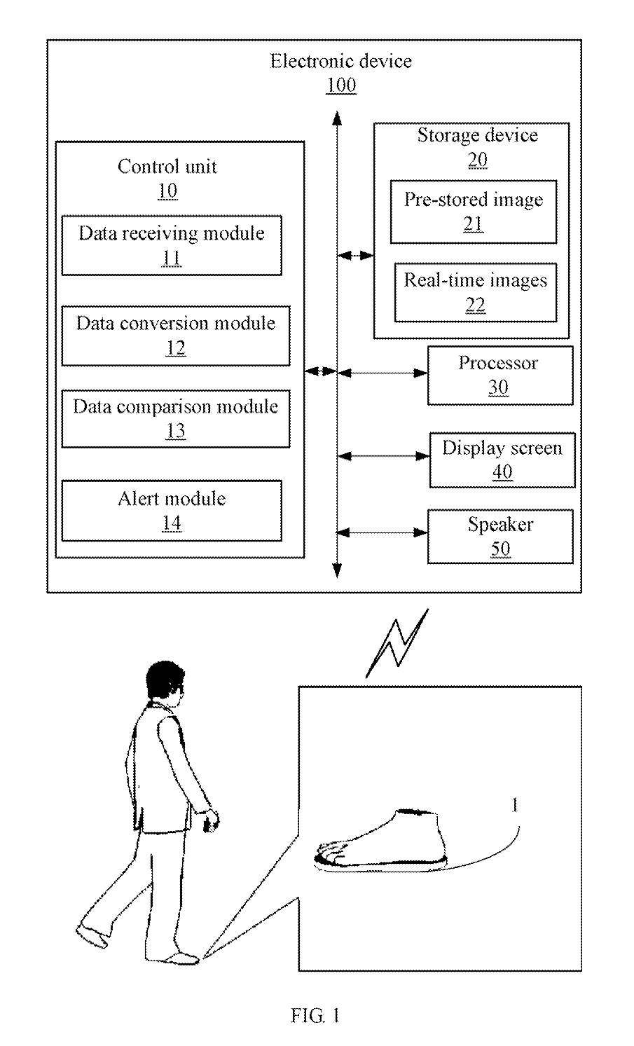

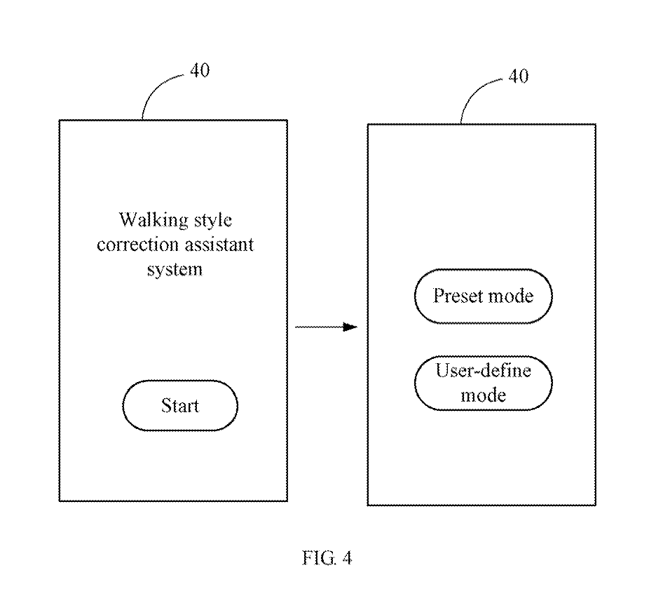

FIG. 1 is one embodiment of function modules of an electronic device 100 comprising a control unit for assisting correction of walking styles of users (“walking style correction assistant system” as shown in FIG. 4). The electronic device 100 further comprises a storage device 20, a processor 30, a display screen 40, and a speaker 50. The electronic device 100 is a portable mobile device, such as a mobile phone or a personal digital assistant.

As shown in FIG. 1, an insole 1 is put in each of a pair of shoes of the user. The insole 1 includes a pressure sensor (not shown). The pressure sensor detects real-time data in relation to pressures on the plantar fascia of the user (hereinafter “the plantar pressures”) when the user is walking. A wireless communication module, such as a wireless network module or a BLUETOOTH module, is installed in the insole 1, to provide communication with the electronic device 100.

The control unit 10 generates a real-time image 22 in relation to distributions of real-time pressures on the plantar fascia of the user (hereinafter, “the real-time plantar pressure distributions”) according to the real-time data, compares the real-time image 22 with a pre-stored image 21 in relation to plantar pressure distributions of a standardized and correct walking style, determines if a walking style of the user needs to be adjusted according to a comparison result of the real-time image 22 and the pre-stored image 21, and prompts the user to adjust his walking style via an output device, such as the display device 40 or the speaker 50 of the electronic device 100.

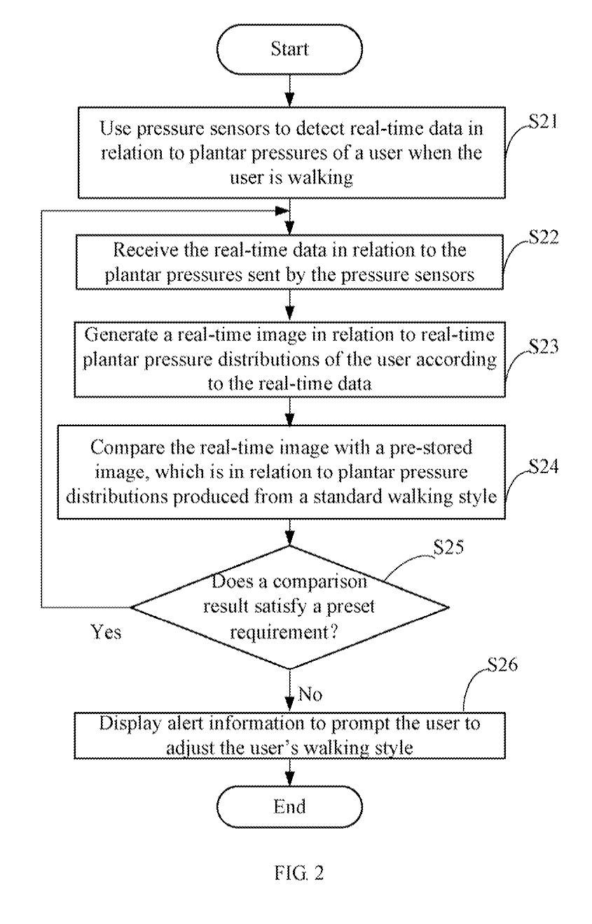

In this embodiment, the control unit 10 includes a data receiving module 11, a data conversion module 12, a data comparison module 13, and an alert module 14. The modules 11-14 may include computerized code in the form of one or more programs that are stored in the storage device 20. The computerized code includes instructions to be processed by the processor 30 to provide the aforementioned functions of the control unit 10. A description of the functions of the modules 11-14 is illustrated in FIG. 2. The storage device 20 may be a cache or a dedicated memory, such as an EPROM, a hard disk driver (HDD), or flash memory.

FIG. 2 is a flowchart of one embodiment of a method for assisting correction of a walking style of a user using the electronic device 100. Depending on the embodiment, additional steps may be added, others removed, and the ordering of the steps may be changed.

In step S21, when the user of the electronic device 100 is walking, pressure sensors installed within insoles in shoes of the user, detect real-time data in relation to plantar pressures of the user. In this embodiment, the real-time data in relation to the plantar pressures of the user includes position coordinates and a pressure value of each of the plantar pressures. The pressure sensors send the real-time data in relation to the plantar pressures to the electronic device 100 via a wireless module, such as a wireless network module or a BLUETOOTH module. The pressure sensors may be capacitive type sensors, resistance type sensors, or voltage type sensors, which are well known to one of ordinary skill in the art.

In step S22, the data receiving module 11 receives and stores the real-time data in relation to the plantar pressures of the user into the storage device 20.

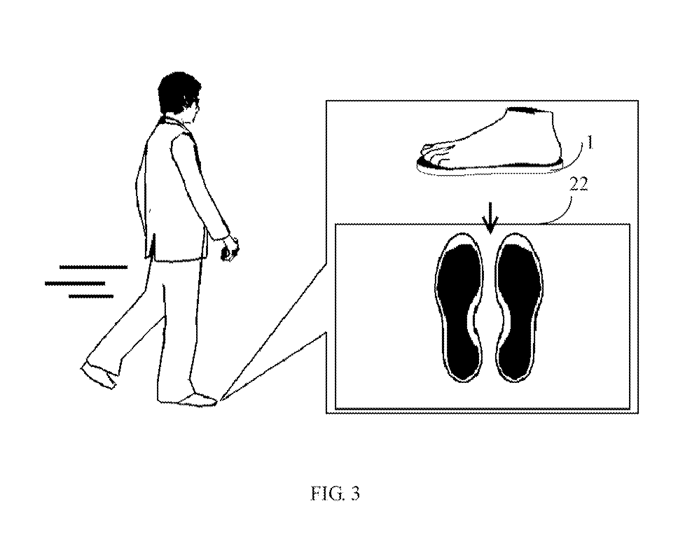

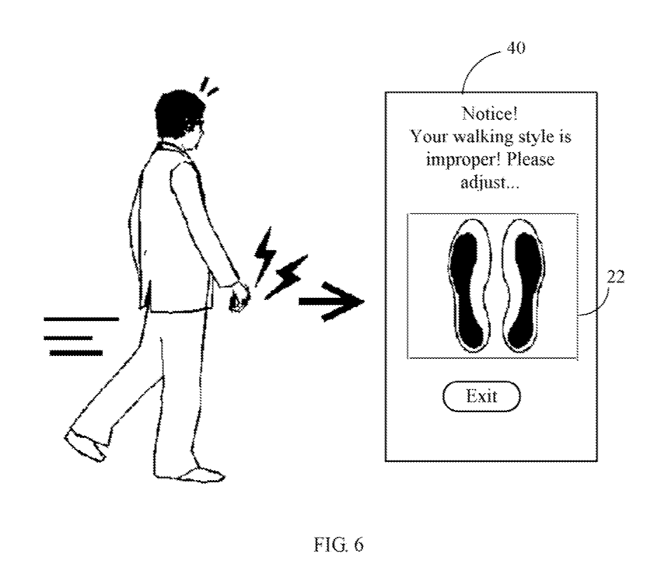

In step S23, the data conversion module 12 generates a real-time image 22 in relation to real-time plantar pressure distributions of the user according to the real-time data. For example, the data conversion module 12 may convert the pressure value of each of the plantar pressures to a gray value, and produce pixel points according to the position coordinates and the gray values of the plantar pressures to form the real-time image 22 (as shown in FIG. 3 and FIG. 6).

In step S24, the data comparison module 13 compares the real-time image 22 with a pre-stored image 21 stored in the storage device, where the pre-stored image 21 is in relation to plantar pressure distributions produced from a standard walking style. For example, the data comparison module 13 compares a gray value of each pixel point in the real-time image 22 with a gray value of a corresponding pixel point in the pre-stored image 21.

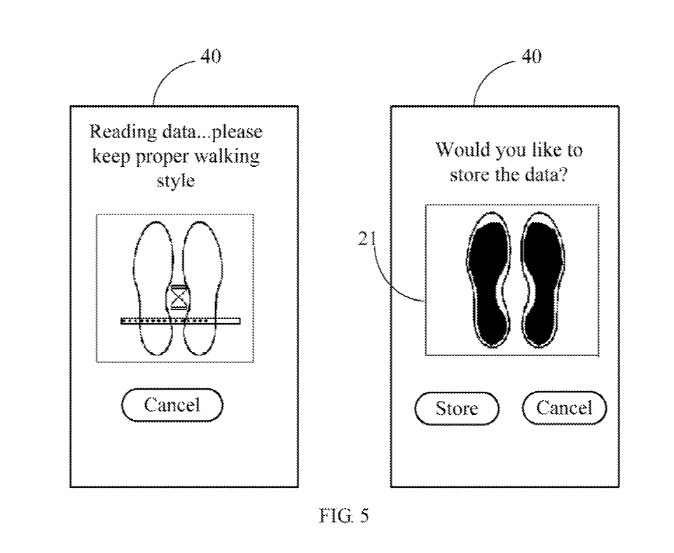

In this embodiment, the pre-stored image 21 may be produced under a user-defined mode or a preset mode. For example, as shown in FIG. 4, when the user starts the function for assisting correcting walking styles, such as by opening the “walking style correction assistant system,” the icons of the preset mode and the user-defined mode are displayed on the display screen 40 for the user to make a selection. If the user selects the preset mode, the pre-stored image 21 is an image provided by a provider of the “walking style correction assistant system,” and is already stored in the storage device 20. If the user selects the user-defined mode, the control unit 10 utilizes the modules 11-14 to generate the pre-stored image 21 (as shown in FIG. 5) when the user keeps a proper walking style. The generation process of the pre-stored image 21 is similar to the generation process of the real-time image 22 described above.

In step S25, the data comparison module 13 determines if a comparison result satisfies a preset requirement. For example, in this embodiment, the data comparison module 13 determines if the real-time image 22 has a preset number of pixel points, where a gray value of each of the preset number of pixel points in the real-time image 22 equals a gray value of a corresponding pixel point in the pre-stored image 21. In another embodiment, the data comparison module 13 may determine if the real-time image 22 has the preset number of pixel points, where the gray value of each of the preset number of pixel points in the real-time image 22 falls within an allowable value range of the gray value of the corresponding pixel point in the pre-stored image 21. If the comparison result satisfies the preset requirement, the data comparison module 13 determines that the user's walking style is proper, the procedure returns to step 22. Otherwise, if the comparison result does not satisfy the preset requirement, the procedure goes to step S26.

In step S26, the alert module 14 displays alert information to prompt the user to adjust the user's walking style. For example, as shown in FIG. 6, the alert module 14 may display the real-time image 22 and an alert message “Notice! Your walking style is improper! Please adjust . . . ”, on the display screen 40, and outputs the alert message via the speaker 50.

Although certain inventive embodiments of the present disclosure have been specifically described, the present disclosure is not to be construed as being limited thereto. Various changes or modifications may be made to the present disclosure without departing from the scope and spirit of the present disclosure.

What is claimed is:

1. A method for assisting correction of walking styles being performed by execution of instructions by a processor of an electronic device, the method comprising:
receiving real-time data in relation to plantar pressures of a user, wherein the real-time data is detected by pressure sensors that are installed within insoles in a pair of shoes of the user;
generating a real-time image in relation to real-time plantar pressure distributions of the user according to the real-time data;
comparing the real-time image with a pre-stored image stored in a storage device and determining if a comparison result satisfies a preset requirement, wherein the pre-stored image is in relation to standard plantar pressure distributions produced from a standard walking style; and
displaying alert information via an output device to prompt the user to adjust the user's walking style, if the comparison result does not satisfy the preset requirement.

2. The method of claim 1, wherein the real-time data in relation to the plantar pressures comprises position coordinates and a pressure value of each of the plantar pressures.

3. The method of claim 2, wherein the real-time image is generated by converting the pressure value of each of the plantar pressures to a gray value, and producing pixel points according to the position coordinates and the gray values of the plantar pressures.

4. The method of claim 1, wherein the preset requirement is defined as the real-time image having a preset number of pixel points, and a gray value of each of the preset number of pixel points in the real-time image equaling a gray value of a corresponding pixel point in the pre-stored image.

5. The method of claim 1, wherein the preset requirement is defined as the real-time image having a preset number of pixel points, and a gray value of each of the preset number of pixel points in the real-time image falling within an allowable value range of the gray value of the corresponding pixel point in the pre-stored image.

6. The method of claim 1, wherein each of the insoles or each of the pair of shoes is installed with a wireless communication module to provide communication and data transmission with the electronic device.

7. The method of claim 6, wherein the wireless communication module is a wireless network module or a BLUETOOTH module.

8. A non-transitory medium storing a set of instructions, the set of instructions capable of being executed by a processor of an electronic device to perform a method for assisting correction of walking styles, the method comprising:
receiving real-time data in relation to plantar pressures of a user, wherein the real-time data is detected by pressure sensors that are installed within insoles put in a pair of shoes of the user;
generating a real-time image in relation to real-time plantar pressure distributions of the user according to the real-time data;
comparing the real-time image with a pre-stored image stored in a storage device and determining if a comparison result satisfies a preset requirement, wherein the pre-stored image is in relation to standard plantar pressure distributions produced from a standard walking style; and
displaying alert information via an output device to prompt the user to adjust the user's walking style, if the comparison result does not satisfy the preset requirement.

9. The medium of claim 8, wherein the real-time data in relation to the plantar pressures comprises position coordinates and a pressure value of each of the plantar pressures.

10. The medium of claim 9, wherein the real-time image is generated by converting the pressure value of each of the plantar pressures to a gray value, and producing pixel points according to the position coordinates and the gray values of the plantar pressures.

11. The medium of claim 8, wherein the preset requirement is defined as the real-time image having a preset number of pixel points, and a gray value of each of the preset number of pixel points in the real-time image equaling a gray value of a corresponding pixel point in the pre-stored image.

12. The medium of claim 8, wherein the preset requirement is defined as the real-time image having a preset number of pixel points, and a gray value of each of the preset number of pixel points in the real-time image falling within an allowable value range of the gray value of the corresponding pixel point in the pre-stored image.

13. The medium of claim 8, wherein each of the insoles or each of the pair of shoes is installed with a wireless communication module to provide communication and data transmission with the electronic device.

14. An electronic device, comprising:
a storage device;
a processor; and
one or more programs stored in the storage device and being executable by the processor, the one or more programs comprising instructions:
receive real-time data in relation to plantar pressures of a user, wherein the real-time data is detected by pressure sensors that are installed within insoles put in a pair of shoes of the user;
generate a real-time image in relation to real-time plantar pressure distributions of the user according to the real-time data;
compare the real-time image with a pre-stored image stored in a storage device and determine if a comparison result satisfies a preset requirement, wherein the pre-stored image is in relation to standard plantar pressure distributions produced from a standard walking style; and
display alert information via an output device to prompt the user to adjust the user's walking style, if the comparison result does not satisfy the preset requirement.

15. The device of claim 14, wherein the real-time data in relation to the plantar pressures comprises position coordinates and a pressure value of each of the plantar pressures.

16. The device of claim 15, wherein the real-time image is generated by converting the pressure value of each of the plantar pressures to a gray value, and producing pixel points according to the position coordinates and the gray values of the plantar pressures.

17. The device of claim 14, wherein the preset requirement is defined as the real-time image having a preset number of pixel points, and a gray value of each of the preset number of pixel points in the real-time image equaling a gray value of a corresponding pixel point in the pre-stored image.

18. The device of claim 14, wherein the preset requirement is defined as the real-time image having a preset number of pixel points, and the gray value of each of the preset number of pixel points in the real-time image falling within an allowable value range of the gray value of the corresponding pixel point in the pre-stored image.

19. The device of claim 14, wherein each of the insoles or each of the pair of shoes is installed with a wireless communication module to provide communication and data transmission with the electronic device.

20. The device of claim 14, wherein the wireless communication module is a wireless network module or a BLUETOOTH module.
